(19) United States
(12) Patent Application Publication
(10) Pub. No.: US 2020/0306280 A1
(43) Pub. Date: Oct. 1, 2020

(54) Enhancing medicinal properties of Hemp, Cannabis, and Cannabidiol (“CBD”) with Rhamnolipid

(71) Applicant: Keith DeSanto, St Petersburg, FL (US)

(72) Inventor: Keith DeSanto, St Petersburg, FL (US)

(21) Appl. No.: 16/433,199

(22) Filed: Jun. 6, 2019

Related U.S. Application Data
(60) Provisional application No. 62/824,644, filed on Mar. 27, 2019.

(51) Int. Cl. A61K 31/7032; A61K 36/185; A61K 31/05

(52) U.S. Cl. CPC A61K 31/7032; A61K 31/05; A61K 36/185

(57) ABSTRACT

Rhamnolipid has the ability to supercharge the effectiveness of Hemp, Cannabis, and Cannabidiol (“CBD”). This is caused by rhamnolipid enhancing the effectiveness of the medicinal properties found in Hemp, Cannabis, and CBD. Rhamnolipid also has antifungal and antibacterial properties in humans when ingested and in food processing. With rhamnolipid infused with Hemp, Cannabis, and CBD, the end user can receive a supercharged medicinal effect with added antifungal and antibacterial properties.

Rhamnolipid has the ability to supercharge the effectiveness of Hemp, Cannabis, and Cannabidiol (“CBD”). This is caused by rhamnolipid enhancing the effectiveness of the medicinal properties found in Hemp, Cannabis, and CBD. Rhamnolipid also has antifungal and antibacterial properties in humans when ingested and in food processing. With rhamnolipid infused with Hemp, Cannabis, and CBD, the end user can receive a supercharged medicinal effect with added antifungal and antibacterial properties.

Rhamnolipid has the ability to supercharge the effectiveness of Hemp, Cannabis, and Cannabidiol (“CBD”). This is caused by rhamnolipid enhancing the effectiveness of the medicinal properties found in Hemp, Cannabis, and CBD. Rhamnolipid also has antifungal and antibacterial properties in humans when ingested and in food processing. With rhamnolipid infused with Hemp, Cannabis, and CBD, the end user can receive a supercharged medicinal effect with added antifungal and antibacterial properties.

1. Rhamnolipid mixed and/or infused with Hemp, Cannabis, and Cannabidiol (“CBD”) to supercharge and enhance the medicinal properties of Hemp, Cannabis, and CBD.

2. Using claim 1, where the Rhamnolipid has antibacterial and antifungal properties.

3. Using claim 1, where the Hemp, Cannabis, or CBD is ingested or applied topically with creams, oils, sprays, or other application methods.
